(19) United States
(12) Patent Application Publication
(10) Pub. No.: US 2007/0100148 A1
(43) Pub. Date: May 3, 2007

(54) PROCESS FOR PREPARING ANASTROZOLE

(76) Inventors: Veerender Murki, Secunderabad 500 015, A.P. (IN); Venkata Raghavendra Acharyulu Palle, Hyderabad 500 050, A.P. (IN); Pandurang Prasad Panchbhai, Sholapur 413 402, Maharashtra (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/554,661

(22) Filed: Oct. 31, 2006

Related U.S. Application Data
(60) Provisional application No. 60/745,881, filed on Apr. 28, 2006.

(30) Foreign Application Priority Data
Oct. 31, 2005 (IN) 1589/CHE/2005

(51) Int. Cl. C07D 249/08

(52) U.S. Cl. 548/262.2

(57) ABSTRACT

A process for preparing anastrazole.

### INTRODUCTION TO THE INVENTION

The present invention relates to a process for the preparation of anastrozole and intermediates thereof. More particularly it relates to a process for the preparation of the 2-[3-bromomethyl-5-(cyano-dimethyl-methyl)-phenyl]-2-methyl-propionitrile compound of Formula II and its conversion to anastrozole. It also relates to a process for purification of anastrozole to remove process and structure related impurities.

Anastrozole is chemically known as 1,3-Benzenediacetonitrile, α,α,α′,α′-tetramethyl-5-(1H-1,2,4-triazol-1-ylmethyl) and is represented by structural Formula I.

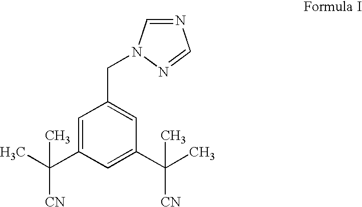

Anastrozole is a non-steroidal aromatase inhibitor used for treatment of breast cancer and is commercially available in the market under the brand name ARIMIDEX® in the form of tablets containing 1 mg of anastrozole.

U.S. Pat. No. RE36,617 and European Patent No. 0296749 disclose anastrozole, pharmaceutical acceptable salts, compositions containing anastrozole and their use in the treatment of breast cancer. They also disclose a process for the preparation of anastrozole, in which the 2-[3-bromomethyl-5-(cyano-dimethyl-methyl)phenyl]-2-methyl-propionitrile compound of Formula II is prepared by using benzoyl peroxide as a catalyst for bromination, its conversion to anastrozole and purification using flash chromatography.

U.S. Patent Application Publication No.2006/0035950 A1 discloses a process for the preparation of anastrozole and intermediates thereof, including preparing the 2-[3-bromomethyl-5-cyano-dimethyl-methyl)-phenyl]-2-methyl-propionitrile compound of Formula II using benzoyl peroxide or UV light as a catalyst for bromination.

International Application Publication No. 2005/105762 A1 discloses a process for the preparation of anastozole, in which 2-[3-bromomethyl-5-cyano-dimethyl-methyl)phenyl]-2-methyl-propionitrile compound of Formula II is prepared using m-chlorobenzoyl peroxide as a catalyst for bromination. The final product obtained was purified by formation of an acid addition salt and recrystallization of acid addition salts.

The foregoing processes suffer from serious disadvantages such as low yields of anastrozole and use of hazardous or expensive reagents, rendering the processes unsuitable for industrial scale manufacturing.

There remains a need for a safe and efficient industrial scale process for preparing anastrozole that is free of the above-mentioned problems.

According to the present invention there is provided a convenient process for the preparation of anastrozole and its intermediates with desired purity and yield by using preparation techniques, which are simple, ecofriendly, cost-effective, robust and well suited for employment on an industrial scale.

### SUMMARY OF THE INVENTION

The present invention relates to a process for the preparation of anastrozole and intermediates thereof.

In one aspect, the present invention relates to a process for the preparation of anastrozole of Formula I, comprising the steps of:

i) reacting 2-2′-(5-methyl-1,3-phenylene)-di-(2-methylpropiononitrile) compound of Formula III with a suitable brominating agent in the presence of a free radical initiator to give 2-[3-bromomethyl-5-(cyano-dimethyl-methyl)-phenyl]-2-methyl-propionitrile of Formula II; and

ii) condensation of 2-[3-bromomethyl-5-(cyano-dimethyl-methyl)-phenyl]-2-methyl-propionitrile of Formula II with 1,2,4-triazole of Formula VI or its metal salt, in the presence of a solvent, to give anastrozole of Formula I.

In another aspect, the invention provides a process for purification of anastrozole comprising crystallizing the anastrozole from its solution in isopropanol, including combining the solution with water.

Yet another aspect of the present invention provides crystalline anastrozole characterized by its X-ray powder diffraction (“XRPD”) pattern and/or its differential scanning calorimetry (“DSC”) curve.

In an embodiment, the invention includes a process for preparing anastrazole, comprising reacting 2,2′-(5-methyl-1,3-phenylene)-di-(2-methylpropionitrile) with a brominating agent, in the presence of a free radical initiator, to form 2-[3-bromomethyl-5-cyano-dimethyl-methyl)-phenyl]-2-methyl-propionitrile.

In another embodiment, the invention includes a process for purifying anastrozole, comprising combining a solution of anastrozole in isopropanol with water, to crystallize anastrozole.

In yet another embodiment, the invention includes a process for preparing anastrazole, comprising reacting 2,2′-(5-methyl-1,3-phenylene)-di-(2-methylpropionitrile) with a brominating agent, in the presence of azobis(isobutyronitrile), to form 2-[3-bromomethyl-5-cyano-dimethyl-methyl)-phenyl]-2-methyl-propionitrile, then reacting with a salt of 1,2,4-triazole, in a solvent comprising N,N-dimethylacetamide, to form anastrozole.

In a further embodiment, the invention includes a process for purifying anastrozole, comprising passing a solution of anastrozole through a silica gel bed.

### BRIEF DESCRIPTION OF THE DRAWINGS

FIG. 1 is an X-ray powder diffraction pattern of anastrozole prepared according to Example 3.

FIG. 2 is a differential scanning calorimetry curve of anastrozole prepared according to Example 3.

FIG. 3 is a thermogravimetric analysis curve of anastrozole prepared according to Example 3.

### DETAILED DESCRIPTION OF THE INVENTION

The present invention relates to the preparation of anastrozole and intermediates thereof.

In one aspect, the present invention relates to a process for the preparation of the anastrozole compound of Formula I, comprising the steps of:

i) reacting 2-2′-(5-methyl-1,3-phenylene)di(2-methylpropiononitrile) of Formula III with a suitable brominating agent in the presence of a free radical initiator to give 2-[3-bromomethyl-5-(cyano-dimethyl-methyl)-phenyl]-2-methyl-propionitrile of Formula II; and

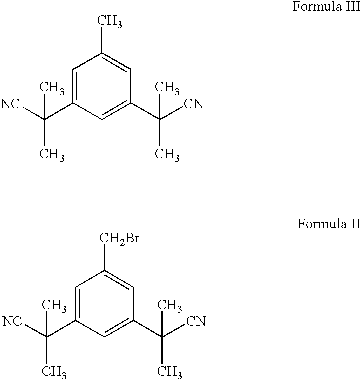

ii) condensation of 2-[3-bromomethyl-5-(cyano-dimethyl-methyl)-phenyl]-2-methyl-propionitrile of Formula II with 1,2,4-triazole of Formula VI or its metal salt in the presence of a solvent, to give anastrozole of Formula I.

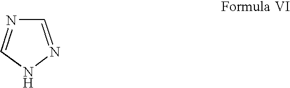

Step i) involves reaction of 2-2′-(5-methyl-1,3-phenylene) di (2-methylpropiononitrile) of Formula III with a suitable brominating agent in the presence of a free radical initiator to give 2-[3-bromomethyl-5-cyano-dimethyl-methyl)-phenyl]-2-methyl-propionitrile of Formula II.

Suitable brominating agents include but are not limited to N-bromosuccinimide, bromine gas, bromine in acetic acid, aqueous hydrobromic acid and the like.

Free radical initiators are substances that can produce radical species under mild conditions. Radical initiators that can also be used in step i) include but are not limited to 2-(1-Carbamimidoyl-1-methyl-ethylazo)-2-methyl-propionamidine and the like.

In an embodiment the free radical initiator azobis(isobutyronitrile), or “AIBN,” is used as a catalyst in the process of the present invention. AIBN is an efficient source of radicals and is used in the production of bromine radicals. AIBN is safe to use in commercial production because no oxygenated residue are produced.

The free radical initiator, such as AIBN, can be used in the process in ratios of about 0.01 to about 0.2 moles, per mole of 2-2′-(5-methyl-1,3-phenylene)di(2-methylpropiononitrile) of Formula III.

Suitable solvents that can be used in the above reaction include but are not limited to: ketonic solvents such as acetone, ethyl methyl ketone, methyl isobutyl ketone and the like; esters such as ethyl acetate, n-propyl acetate, n-butyl acetate, t-butyl acetate and the like; nitrile solvents such as acetonitrile, priopionitrile and the like; halogenated solvents such as chloroform, dichloromethane and the like; or mixtures thereof or their combinations with water in various proportions. In an embodiment dichloromethane is used as a solvent for bromination.

Suitable temperatures for conducting the reaction may range from about −30° C. to about 100° C., or about 40° C. to about 50° C.

2-[3-bromomethyl-5-(cyano-dimethyl-methyl)-phenyl]-2-methyl-propionitrile of Formula II obtained by the process of present invention may be purified by recrystallization in a suitable solvent, if desired.

Suitable solvents which can be used for crystallization or precipitation include but are not limited to: hydrocarbons such as n-hexane, cyclohexane, heptane and the like; aromatic solvents such as benzene, toluene and the like; alcohol solvents such as methanol, ethanol, isopropanol, and the like; esters such as ethyl acetate, methyl acetate, isopropyl acetate and the like; and halogenated solvents such as dichloromethane, and the like.

Step ii) involves condensation of 2-[3-bromomethyl-5-(cyano-dimethyl-methyl)phenyl]-2-methyl-propionitrile of Formula II with 1,2,4-triazole or a metal salt thereof in the presence of a solvent to give the compound of Formula I.

Suitable solvents that can be used in the condensation reaction include but are not limited to dimethylacetamide (DMA), dimethylsulfoxide (DMSO), acetonitrile and the like.

Suitable metal salts of 1,2,4-triazole for use in the condensation reaction include but are not limited to the sodium, potassium, and aluminum salts, and the like.

Suitable temperatures for conducting the reaction range from about 20° C. to about 70° C., or about 50° C. to about 65° C.

After reaction completion, the reaction mixture is quenched with water and extracted with a suitable water immiscible solvent.

Suitable solvents that can be used for extraction of anastrozole include but are not limited to: chlorinated solvents such as dichloromethane, dichloroethane, chloroform, and the like; esters such as ethyl acetate, methyl acetate and the like; ethers such as diethyl ether, diisopropyl ether, and the like; and hydrocarbons such as n-hexane, cyclohexane, heptane and the like.

The organic layer comprising anastrozole is treated with a suitable inorganic acid to extract the product into the aqueous layer.

Suitable acids that can be used for extraction include but are not limited to inorganic acids such as hydrochloric acid, hydrobromic acid and the like.

The aqueous layer comprising anastrozole is treated with a suitable inorganic base and the product is extracted with a suitable solvent.

Suitable bases that can be used include but are not limited to sodium or potassium hydroxide, bicarbonate or carbonate, ammonia and the like.

Suitable solvents which can be used for extraction of anastrozole include but are not limited to chlorinated solvents such as dichloromethane, dichloroethane, chloroform, and the like;

The organic layer comprising anastrozole may be used directly in the next processing step or it can be concentrated to form a residue.

Anastrazole obtained at this stage may contain some isomeric and structure related impurities. In an embodiment of the present invention, there is also provided a process for the purification of anastrozole to reduce the concentrations of isomeric impurities and structural related impurities, which process comprises:

a) providing a solution of crude anastrozole in a suitable solvent;

b) passing the solution of step a) through a silica gel bed; and

c) recovering purified anastrozole.

The step of providing a solution of crude anastrozole includes dissolving crude anastrozole in suitable solvent or obtaining a solution comprising crude anastrozole from a previous processing step such as the synthesis of anastrozole.

Suitable solvents which can be used for providing a solution of crude anastrozole include but are not limited to: chlorinated solvents such as dichloromethane, dichloroethane, chloroform, and the like; esters such as ethyl acetate, methyl acetate and the like; ethers such as diethyl ether, diisopropyl ether, and the like; and hydrocarbons such as n-hexane, cyclohexane, heptane and the like.

Step b) involves passing the solution obtained from step a) through a silica gel bed. The silica gel bed is prepared by filling a column with silica gel.

Silica gel that is used for filing the column can have a particle size range from about 240-400 mesh, 100-200 mesh, or 60-120 mesh. The quantity of silica gel used can range from about 4 to 10 times, or about 4 to 6 times, the weight of the starting anastrozole in solution.

The column may be made from stainless steel, glass, or other inert materials and the length and diameter of the column can easily be determined by one skilled in the art, depending on the quantities of material to be processed and the levels of contained impurities.

Suitably, synthetic resins may be used instead of silica gel to remove impurities in the said process. Useful synthetic resins include divinylbenzene-styrene copolymers or copolymers of divinylbenzene, styrene and other derivatives of these having aliphatic and/or aromatic moieties comprising from 2 to 18 carbon atoms, or having substituted halogen atoms chlorine, fluorine or bromine, or copolymers of divinylbenzene and styrene with surface grafted moieties that are aliphatic or aromatic containing two or more carbon atoms and/or having substituted halogen atoms chlorine, fluorine or bromine, a resin based on one or combination of natural polymers, derivatized or not, such as for example, agarose, dextran or cellulose, a resin based on polymethacrylate matrix, or its combination with other acrylate polymers, prepared by cross-linking of monomers, with or without grafted moieties that are aliphatic or aromatic containing two or more carbon atoms with or without substituted halogen atoms chlorine, fluorine or bromine.

Pure anastrazole is recovered by concentrating the eluant obtained from the previous step using suitable techniques.

Suitable techniques which can be used for a distillation concentration include distillation using a rotational evaporator device such as a Buchi Rotavapor, spray drying, agitated thin film drying (“ATFD”), and the like.

Distillation of the solvent may be conducted under a vacuum, such as below about 100 mm Hg to below about 600 mm Hg, at elevated temperatures such as about 20° C. to about 70° C. Any temperature and vacuum conditions can be used as long as there is no increase in the impurity levels of the product.

In another aspect, the present invention relates to a process for the purification of anastrozole comprising crystallizing from a mixture comprising isopropanol and water. An embodiment of this process includes:

1 ) providing a solution of anastrozole in isopropyl alcohol;

2) adding water to the solution of step 1); and

3) recovering crystalline anastrozole.

The step of providing a solution of anastrozole includes dissolving anastrozole in isopropyl alcohol or obtaining a solution comprising anastrozole and isopropyl alcohol from a previous processing step.

Any form of anastrozole is acceptable for providing the solution, such as any crystalline or amorphous form of anastrozole.

The concentration of anastrozole in the isopropyl alcohol is not critical as long as sufficient isopropyl alcohol is employed to ensure total dissolution. The amount of isopropyl alcohol employed is ordinarily kept as low as possible to avoid excessive product loss during crystallization and isolation. The quantity of isopropyl alcohol used for the isolation of the crystalline form of anastrozole frequently is about 0.5 to about 5 times the weight of anastrozole.

The solution can be prepared at any temperatures up to the boiling point of the solvent. Frequently, the solution will be prepared at an elevated temperature for enhanced solute concentration.

The solution can be optionally treated with activated charcoal to enhance the color of the compound and filtered through an inert medium such as through a flux calcined diatomaceous earth (“Hyflow”) bed to remove the carbon.

The solution can optionally be filtered by passing through paper, glass fiber, or other membrane material or a clarifying agent such as celite. Depending upon the equipment used and the concentration and temperature of the solution, the filtration apparatus may need to be heated to avoid premature crystallization.

Step 2) involves adding water to the solution of step 1).

The quantity of water that can be used in the process of step 2) frequently is from about 3 to about 15 times the weight of anastrozole.

The mixture may be maintained further at temperatures below about 40° C. to about 45° C., for a period of time as required for a more complete isolation of the product. The exact cooling temperature and time required for complete crystallization can be readily determined by a person skilled in the art and will also depend on parameters such as concentration and temperature of the solution or slurry.

In an alternative embodiment, the solution of step 1) can be added to the water.

Step 3) involves recovering the crystalline anastrozole.

The solid can be isolated by conventional techniques such as filtering, decanting, centrifuging and the like, or by filtering under an inert atmosphere using gases such as for example nitrogen and the like.

The wet cake obtained may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods until the desired product purity is obtained; frequently times from about 1 to 20 hours, or longer, are used.

Anastrozole obtained by the process of present invention has low levels of one or more of the following structural related impurities:

a) 1,3-dibenzenediacetonitrile, 5-methyl of Formula VII (“Impurity A”);

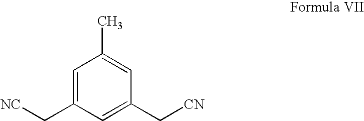

b) 1,3-dibenzenediacetonitrile, 5-methyl, α,α,α′,α′-pentamethyl of Formula II (“Impurity B”);

c) 1,3-dibenzenediacetonitrile, 5-(bromomethyl)-α,α,α′,α′-tetramethyl of Formula III (“Impurity C”);

d) 1,3-dibenzenediacetonitrile, α,α,α′,α′-tetramethyl-5-(4H-1,2,4-triazol-4-ylmethyl) of Formula IV (“Impurity D”); and

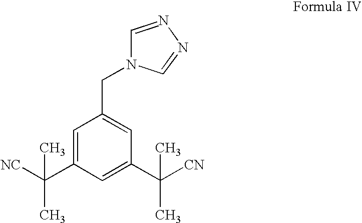

e) 2-[3-(bis-[1,2,4]-triazol-1-yl-methyl)-5-(cyano-dimethyl-methyl)-phenyl]-2-methyl-propionitrile of Formula V (“Impurity E”).

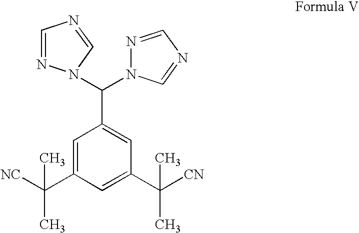

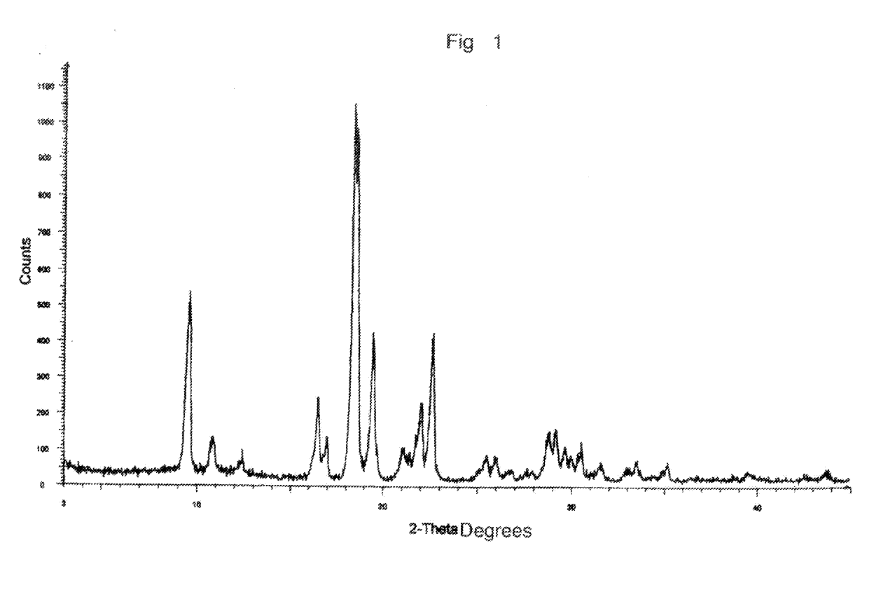

Crystalline anastrozole obtained in the process of present invention is characterized by its XRPD pattern, substantially in accordance with the pattern of FIG. 1. All XRPD data reported herein were obtained using Cu Kα radiation, having the wavelength 1.541 Å and were obtained using a Bruker Axe D8 Advance Powder X-ray Diffractometer.

Crystalline anastrozole is characterized by an XRPD diffraction pattern comprising characteristic peaks approximately at about 9.6, 16.5, 18.4, 18.6, 19.5, 22.1, 22.7, 26.1, 28.8, and 29.2, ±0.2 degrees two theta.

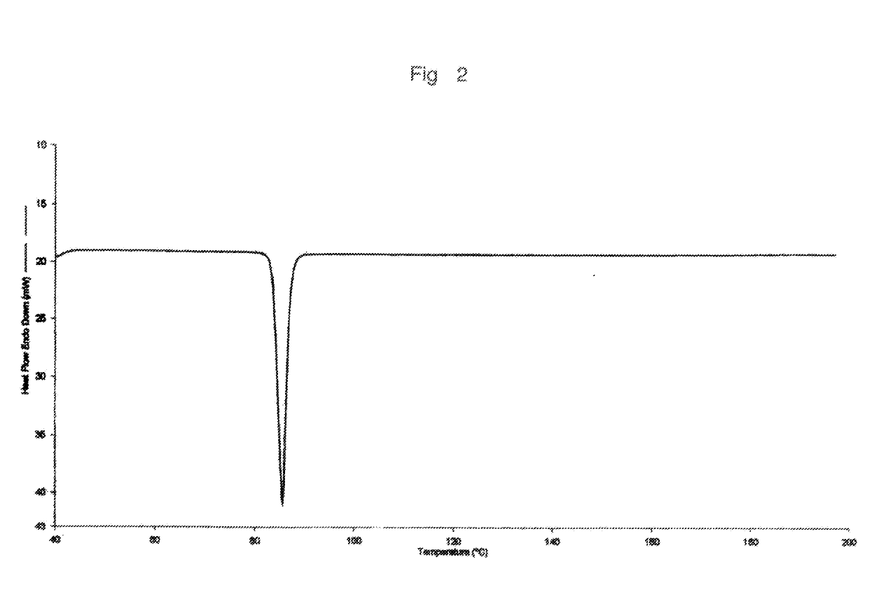

Crystalline anastrozole of the present invention has a characteristic differential scanning calorimetric curve substantially in accordance with FIG. 2, having an endothermic peak at about 86° C.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

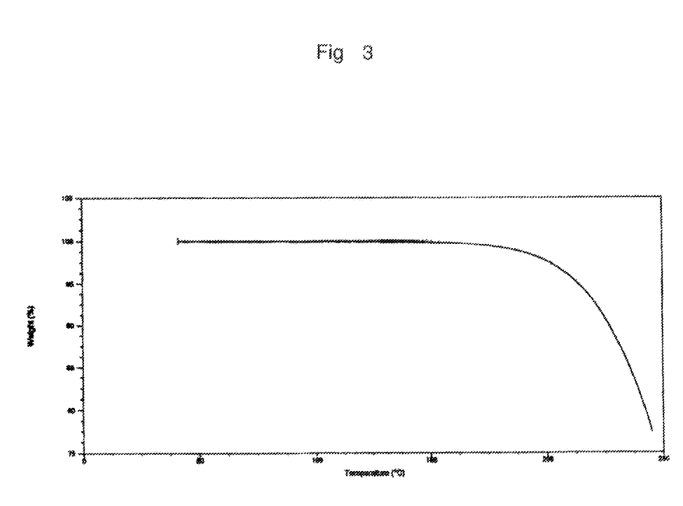

Crystalline anastrozole of the present invention has a characteristic termogravimetric curve (TGA) corresponding to weight loss of about 0.4% w/w substantially in accordance with FIG. 3, showing that it is in an anhydrous form.

In an embodiment, anastrozole obtained by the process of present invention has particle size of D₉₀ less than about 500 microns, or about 300 microns, or about 100 microns.

The D₁₀, D₅₀ and D₉₀ values are useful ways for indicating a particle size distribution. D₉₀ refers to the value for the particle size for which at least 90 volume percent of the particles have a size smaller than the value given. Likewise D₅₀ and D₁₀ refer to the values for the particle size for which 50 volume percent, and 10 volume percent, of the particles have a size smaller than the value given. Methods for determining D₁₀, D₅₀ and D₉₀ include laser light diffraction, such as using equipment from Malvern Instruments Ltd. (of Malvern, Worcestershire, United Kingdom). There is no specific lower limit for any of the D values.

Anastrozole obtained using the process of the present invention has amount of residual solvent content that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

Anastrozole obtained in this invention contains less than about 100 ppm or less than about 500 ppm of methanol, less than about 100 ppm or less than about 500 ppm of isopropyl alcohol, less than about 100 ppm or less than about 500 ppm of n-hexane, less than about 100 ppm or less than about 500 ppm of dichloromethane, less than about 100 ppm or less than about 500 ppm of acetone, less than about 100 ppm or less than about 500 ppm of N,N-dimethylacetamide, less than about 100 ppm or less than about 500 ppm of methyl isobutyl ketone, less than about 100 ppm or less than about 500 ppm of N,N-dimethyl formamide, and less than about 100 ppm or less than about 500 ppm of toluene.

Certain specific aspects and embodiments of the invention will be explained in more detail with reference to the following examples, which are provided by way of illustration only and should not be construed as limiting the scope of the invention in any manner.

### EXAMPLE 1

### Preparation of 2-[3-bromomethyl-5-cyano-dimethyl-methyl)-phenyl]-2-methyl-propionitrile (Formula II)

1.5 Kg of 2-2′-(5-methyl-1,3-phenylene) di-(2-methylpropiononitrile) and 30 L of dichloromethane were charged into a glass flask followed by stirring for 10 minutes. 1.2 Kg of N-bromosuccinimide was charged followed by charging of 0.022 Kg of azobis(isobutyronitrile) and the reaction solution was stirred for 10 minutes. The resultant reaction mixture was heated to about 40.5° C. for 6 hours followed by cooling to 3° C. The reaction mixture was stirred for about for 30 minutes at 3° C. followed by filtering. The resultant filtrate was taken into a clean and dry glass flask followed by charging of 0.35 Kg of N-bromosuccinimide, 0.022 Kg of azobis(isobutyronitrile) and 2 L of dichloromethane. The resultant reaction mass was stirred for 10 minutes and heated to about 40.5° C. for 24 hours. The reaction mass was cooled to about 3.7° C. for 30 minutes followed by filtering. The resultant filtrate was washed with sodium bisulphite solution (7.5 L of water and 0.75 Kg of sodium bisulphate) followed by separation of the organic layer and distilling at 40° C. under a vacuum of 600 mm Hg. The obtained wet residue was dissolved in 2.25 L of dichloromethane and stirred for 10 minutes, followed by charging of 16 L of n-hexane. The resultant suspension was stirred for 30 minutes followed by filtering the separated solid and washing the solid with 2 L of n-hexane. The solid obtained was suction dried for 2 hours to afford 1.6 Kg of title compound.

### EXAMPLE 2

### Preparation of 1,3-benezenediacetonitrile, α,α,α′,α′-tetramethyl-5-(1H-1,2,4-triazol-1-ylmethyl) (Formula I)

2 Kg of 2-[3-bromomethyl-5-cyano-dimethyl-methyl)-phenyl]-2-methyl-propionitrile and 10 L of N,N-dimethylacetamide were charged in a clean dry reactor followed by stirring for 10 minutes. 0.861 Kg of sodium triazole was charged under nitrogen pressure and the temperature raised to 57.5° C. The obtained solution was maintained for 36 hours at 57.5° C. and then cooled to about 25° C. followed by quenching the reaction by charging the solution to 30 L of water. The resultant reaction solution was extracted with 3×20 L of ethyl acetate followed by separation of organic and aqueous layers. The combined organic layer was distilled at 26.2° C. under a vacuum of −0.6 Kg/cm² to afford a residue, the and the obtained residue was dissolved in 20 L of toluene followed by treatment with 2×2 L of 2N aqueous hydrochloric acid solution and with 2×8 L of water. The aqueous layer was separated and treated with 4×10 L of toluene and the obtained aqueous layer was charged into sodium bicarbonate solution, which was prepared by the dissolution of 4.7 Kg of sodium bicarbonate in 45 L of water. The obtained solution was stirred for 10 minutes and then extracted with 2×20 L of dichloromethane.

A silica gel bed in a stainless steel column filter (2.05 cm diameter and 5.75 cm height) was prepared with 6.4 Kg of 230-400 mesh silica gel and the bed was washed with 4 L of ethyl acetate. The dichloromethane solution from above was passed through the silica gel bed and then the bed was washed with 38 L of ethyl acetate. The combined solution and washing was concentrated at 24.7° C. under a vacuum of −0.6 Kg/cm² and cooled to 30° C. followed by dissolution of the residue in 1.4 L of isopropyl alcohol. The obtained solution was stirred for 10 minutes at 30° C. and 7 L of water were charged. The obtained suspension was stirred for 3 hours followed by filtration through a Nutsche filter and washing the obtained solid with a solution of 0.25 L of isopropyl alcohol and 1 L of water. The resultant solid was dried at 48° C. under vacuum of 690 mm Hg for 3 hours to afford 0.470 Kg of the title compound having a water content by Karl Fischer of 0.13% w/w.

### EXAMPLE 3

### Recrystallisation of 1,3-benzenediacetonitrile, α,α,α′,α′-tetramethyl-5-(1H-1,2,4-triazol-1-ylmethyl) (Formula I)

600 g of the compound of Formula I was dissolved in 12 L of isopropyl alcohol and the temperature raised to 47.5° C. followed by stirring for 5 minutes at 47.5° C. The obtained solution was filtered through a 0.2 μm filter into a glass flask reactor followed by passing 2 L of isopropyl alcohol through the filter into the flask, and concentration at 50° C. under a 645 mm Hg vacuum until no more solvent was distilled. The obtained residue was maintained under vacuum for 30 minutes. 1.2 L of isopropyl alcohol was charged to the residue followed by raising the temperature to 47.5° C. and stirring to afford a clear solution. 6 L of water was charged to the solution and the mixture was cooled to 34.9° C. and the obtained suspension was maintained for 3 hours at 34.9° C. The obtained suspension was filtered through a Nustche filter by applying vacuum and the solid was washed with mixture of isopropyl alcohol (0.1 L) and water (0.5 L ). The resultant solid was dried at 45° C. under vacuum at 710 mm Hg for 3 hours to afford 428 g of the title compound.

Water content by Karl Fischer: 0.06% w/w.

Purity by high performance liquid chromatography (“HPLC”): 100% w/w.

Impurities by HPLC:
Impurity A: Less than 0.002%.
Impurity B: Less than 0.02%.
Impurity C: Less than 0.006%.
Impurity D: Less than 0.008%.
Impurity E: 0.01%.
Unidentified impurities: Not detected.
Total impurities: 0.03%.

Particle size: D₉₀ 33 μm.

We claim:

1. A process for preparing anastrazole, comprising reacting 2,2′-(5-methyl-1,3-phenylene)-di-(2-methylpropionitrile) with a brominating agent, in the presence of a free radical initiator, to form 2-[3-bromomethyl-5-cyano-dimethyl-methyl)-phenyl]-2-methyl-propionitrile.

2. The process of claim 1, wherein a brominating agent comprises N-bromosuccinimide, bromine gas, bromine in acetic acid, or aqueous hydrobromic acid.

3. The process of claim 1, wherein a free radical initiator comprises azobis(isobutyronitrile).

4. The process of claim 1, wherein about 0.01 to about 0.2 moles of free radical initiator, per mole of 2-2′-(5-methyl-1,3-phenylene)-di-(2-methylpropiononitrile), is used.

5. The process of claim 1, wherein reacting is conducted in a solvent comprising dichloromethane.

6. The process of claim 1, further comprising reacting 2-[3-bromomethyl-5-cyano-dimethyl-methyl)-phenyl]-2-methyl-propionitrile with 1,2,4-triazole or a salt thereof, in a solvent comprising N,N-dimethylacetamide, to form anastrozole.

7. The process of claim 6, wherein reacting with 1,2,4-triazole or a salt thereof is conducted at temperatures about 50° C. to about 65° C.

8. A process for preparing anastrazole, comprising reacting 2,2′-(5-methyl-1,3-phenylene)-di-(2-methylpropionitrile) with a brominating agent, in the presence of azobis(isobutyronitrile), to form 2-[3-bromomethyl-5-cyano-dimethyl-methyl)-phenyl]-2-methyl-propionitrile, then reacting with a salt of 1,2,4-triazole, in a solvent comprising N,N-dimethylacetamide, to form anastrozole.

9. The process of claim 8, wherein a brominating agent comprises N-bromosuccinimide, bromine gas, bromine in acetic acid, or aqueous hydrobromic acid.

10. The process of claim 8, wherein about 0.01 to about 0.2 moles of azobis(isobutyronitrile), per mole of 2-2′-(5-methyl-1,3-phenylene)-di-(2-methylpropiononitrile), is used.

11. The process of claim 8, wherein reacting is conducted in a solvent comprising dichloromethane.

12. A process for purifying anastrozole, comprising combining a solution of anastrozole in isopropanol with water, to crystallize anastrozole.

13. The process of claim 12, wherein an amount of water is about 3 to about 15 times the weight of anastrozole in a solution.

14. The process of claim 12, wherein combining comprises adding water to a solution of anastrozole.

15. A process for purifying anastrozole, comprising passing a solution comprising anastrozole through a silica gel bed.

16. The process of claim 15, wherein a weight of silica gel is about 4 to about 10 times a weight of anastrozole in a solution.

17. The process of claim 15, wherein a weight of silica gel is about 4 to about 6 times a weight of anastrozole in a solution.

18. The process of claim 15, wherein a solution comprises anastrozole and dichloromethane.

19. The process of claim 15, wherein a solution comprises anastrozole and dichloromethane, a column is washed with ethyl acetate after passing, and anastrozole is recovered from a mixture comprising a dichloromethane solution and an ethyl acetate wash.

20. The process of claim 19, wherein anastrozole is recovered by removing solvent.
